(19) United States
(12) Patent Application Publication
(10) Pub. No.: US 2006/0105443 A1
(43) Pub. Date: May 18, 2006

(54) Process for obtaining biosynthesized lycopene from bacterial cells and the purified lycopene of the same

(76) Inventors: Yan-Chu Wu, Hsin-Chu Hsien (TW); Yuan-Ting Chen, Hsin-Chu Hsien (TW); Chun-Yen Liu, Hsin-Chu Hsien (TW); Mei-Chiao Wu, Hsin-Chu Hsien (TW); Yi Chen Lu, Hsin-Chu Hsien (TW); Ming-Hsi Chiou, Hsin-Chu Hsien (TW); Chi-Jen Du, Hsin-Chu Hsien (TW)

Correspondence Address: THE MAXHAM FIRM, 750 "B" STREET, SUITE 3100, SAN DIEGO, CA, 92101, US

(21) Appl. No.: 10/989,651

(22) Filed: Nov. 15, 2004

(51) Int. Cl. C12P 23/00

(52) U.S. Cl. 435/170; 435/70.1; 435/67

(57) ABSTRACT

This invention is related to a process for isolating and purifying lycopene, and more particularly to a process for large-scaled isolation and purification of the biosynthesized lycopene from bacterial cells via simple steps with high purity and recovery. The steps in the process of the present invention comprises: isolating a biomass from a fermented broth; treating the isolated biomass with alcohol; extracting the lycopene from the alcohol treated biomass with an organic solvent; removing the solid phase from the biomass/solvent extracting mixture; filtering the liquid-phase extract; crystallizing the lycopene by concentrating the liquid-phase extract; separating the dark red lycopene crystals; re-suspending the separated dark red lycopene crystals with acetone; and separating and drying the washed lycopene crystals. A biosynthesized lycopene purified by the process is also provided.

### BACKGROUND OF THE INVENTION

1. Field of the Invention

This invention is related to a process for isolating and purifying lycopene and more particularly to a process for the large-scaled isolation and purification of biosynthesized lycopene from bacterial cells via simple steps with high purity and recovery. An isolated lycopene purified by the aforementioned process is also provided.

2. Description of Related Art

Lycopene is a natural pigment useful for feed addictives, food addictives, cosmetic manufacturing etc and is a naturally-occurring photochemical abundant in a number of commonly existing plants and microbes. Lycopene is a kind of carotenoid and can be produced from pyruvate and glyceraldehyde after a serial of reduction, dehydration, phosphorylation and other biochemical reactions. It can be further catalyzed into β-carotene, -carotene, -carotene, lutein and zeaxanthin through cyclic reaction catalyzed by different cyclases. Lycopene have been previously recognized as an industrially important natural food coloring agent, because it has high staining power in the red region and processes high safety. Recently, lycopene is proved to be a strong antioxidant which neutralizes free radicals especially those derived from oxygen and also to have the ability inhibiting LDL (low-density lipoprotein) oxidation. This will result in reducing cholesterol levels in the blood and prevent the body from the attack-of free radicals. Clinic researches show that lycopene has the potency to confer the protection against prostate cancer, breast cancer and cardiovascular disease. In addition, preliminary researches also suggest that lycopene may reduce the risk of macular degenerative disease, serum lipid oxidation and cancers of the lung, bladder, cervix and skin. Some of the chemical properties of lycopene responsible for these protective actions are well-documented, see e.g. Giovannucci et al., J. Natl. Cancer Inst. 87(23):1767-1776 (Dec. 6, 1995). Morris wt al., 1994, J. Amer. Med. Assoc. 272(18):1439-1441.

Lycopene is a highly unsaturated aliphatic hydrocarbon with thirteen double bonds on its backbone and has its empirical formula as C₄₀H₅₆ and molecular weight of 536.85. Because of its lipophilic nature, it can be dissolved into oils, chloroform, hexane, benzene, dichloromethane, acetone etc., and can not be dissolved into water, ethanol, methanol or their mixture. Its melting point is about 170-175° C. and is a dark red crystal of needle-like shapes. Lycopene is very sensitive to light, oxygen and alleviated temperature.

Lycopene is abundant in wide variety of fruits, vegetables, fungus, microalgae etc. U.S. Pat. No. 3,097,146 and U.S. Pat. No. 3,3699,74 have disclosed the production of lycopene by fermentation of fungus Blakeslea trispora using hexane extraction and chromatography on alumina. Methods of chemical synthesis of lycopene from commercially available starting materials are also described in U.S. Pat. No. 2,842,599, U.S. Pat. No. 4,105,855 and U.S. Pat. No. 5,208,381. However these methods require extremely high cost of time and lengthy and complicated multiple steps, but with the purity still low. One way to increase the productive capacity of biosynthesized lycopene, as known, is to apply recombinant DNA technology to transfer cloned exogenic genes involved in lycopene synthesis into a host which is suitable for production and further purification. Bacterial hosts transformed with above-said recombinant genes like GGPP synthase, crtB, crtI etc are documented to be capable of producing lycopene. High-yield production of lycopene in engineered microbial hosts always requires optimization of the available isoprenoid precursor pool or balancing the expression of carotenoid genes for efficient transformation. In 2001, Farmer and Liao published that balancing the supply of the intermediate precursors, pyruvate and glyceraldehyde-3-phosphate, would significantly improve lycopene production. And using an engineering regulatory dynamic control circuit (Farmer and Liao 2000), to control the engineered lycopene biosynthesis pathway, significantly enhanced lycopene production while reducing the negative impact caused by metabolic imbalance.

Appropriate induction and regulation during fermentation brings potentially greater economical benefits. The use of this DNA-recombinant host permits control over quality, quantity and the selection of the most appropriate and efficient producer organisms as well. Several patents disclose several gene sequences related to carotenoid biosynthesis pathway for the production of lycopene in genetically engineered organisms like bacteria, fungi or mammalian cells, see e.g. U.S. Pat. No. 5,530,189, U.S. Pat. No. 5,530,188, U.S. Pat. No. 5,429,939 and U.S. Pat. No. 5,304,478. In addition to the advantage of higher enrichment level of lycopene, methods using the aforesaid genetically engineered organisms as the starting material are also capable of avoiding the problem of contamination with other undesired pigments, such as β-carotene in plants.

Isolating lycopene from Mucorales fungi is recently described in EP 1201762A1, within which a simplified process is used to isolate biosynthesized lycopene with an increased level of purity by organic solvent extraction of the mycelium in Mucorales fungi, such as Blakslea, Choanephora or Phycomyces. This prior art, similar to the present invention, comprises the steps of alcohol washing, conditioning by drying and disintegration, organic solvent extracting, concentrating the extract, precipitating the lycopene by adding precipitating agents, filtrating and drying the final purified lycopene. The purity of the purified lycopene via above-mentioned process is 94% according to the spectrometry, but its recovery is about <52 to 73% according to the data disclosed in its embodied examples.

### SUMMARY OF THE INVENTION

The present invention is related to a process for isolation of biosynthesized lycopene from bacteria with and increased purity and high recovery.

The starting material in the present invention is bacteria, but, as described above, it is preferred to adopt genetically engineered bacteria having the capability to produce lycopene. The process for obtaining lycopene from bacterial cells comprises the following steps:
(a) harvesting the bacterial biomass, wherein said bacterial biomass is the derived from culturing the genetically engineered bacteria, which contain lycopene biosynthesis genes, through high-cell density fermentation;
(b) washing the harvested biomass with water or aqueous buffer solution such as dipotassium phosphate aqueous solution and isolating the washed wet biomass;
(c) treating the washed wet biomass with alcohol and isolating the bacterial biomass;
(d) extracting the lycopene from the above-said alcohol-treated bacterial biomass by adding methylene chloride, optionally under the stream of nitrogen or with the existence of antioxidant, to form a biomass/methylene chloride extracting mixture;
(e) removing the solid phase from the biomass/methylene chloride extracting mixture and filtrating the separated liquid-phase extract;
(f) crystallizing the lycopene by concentrating the filtrated liquid-phase extract, and separating the lycopne crystals;
(g) re-suspending the separated, although wet, crystal lycopene in acetone; and
(h) isolating and drying the acetone-treated lycopene crystals;
   The process described here enables the embodiments with the purity above 92%, preferably above 95% and more preferably of above 99% and with the recovery above 75%, preferably above 80% and more preferably of above 87%.

### BRIEF DESCRIPTION OF THE DRAWINGS

FIG. 1 is the flow chart which describes the steps in the process of the present invention.

FIG. 2 depicts the purity of the finally isolated lycopene.

### DETAILED DESCRIPTION OF THE PREFERRED EMBODIMENTS

The present invention is related to a process for the isolation of biosynthesized lycopene with an increased purity and recovery from bacteria. The aforesaid bacteria as the starting material in the present invention is the naturally derived or DNA recombinant bacteria, but it is preferred to adopt genetically engineered bacteria having the capability to produce lycopene.

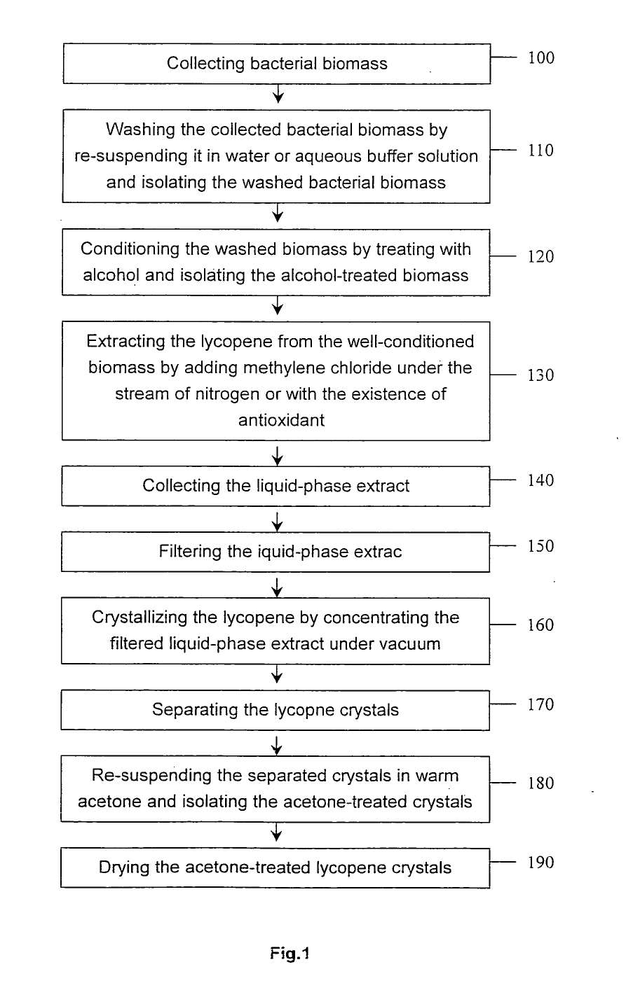

The process for obtaining lycopene from bacterial cells uses the harvested genetically engineered E. coli, which contains lycopene biosynthesis genes, through high-cell density fermentation as the starting material 100, as illustrated in FIG. 1, and other stains of bacteria, naturally derived or DNA recombinant, are also capable to be provided as the starting material, if it is appropriate for the present process. The process of the present invention comprises the steps of:
1. conditioning the collected bacterial biomass by re-suspending the centrifuged or filtered biomass with water or aqueous buffer solution 110 and isolating the washed wet biomass.
2. further conditioning the washed biomass by treating with alcohol and isolating the conditioned bacterial biomass 120;
3. extracting the lycopene from the alcohol-conditioned bacterial biomass by adding methylene chloride under the stream of nitrogen or with the existence of antioxidant 130;
4. collecting 140 and filtering 150 the liquid-phase extract;
5. crystallizing the lycopene by concentrating the filtered liquid-phase extract 160, and separating the lycopne crystals 170;
6. re-suspending the separated, although wet,crystal lycopene in acetone 180;
7. isolating and drying the acetone-treated lycopene crystals 190.

The bacterium used in the present invention is a genetically engineered strain capable of synthesizing lycopene. The aforesaid bacteria are preferred to be Escherichia coli processed by recombinant DNA technology because of a higher concentration of lycopene within the cells and their convenience in regulating the fermentation status and further purification steps.

To collecting the biomass in and after conditioning steps 110 and 120 can be established by both filtration and centrifugation. Rotary filters are commonly used for separating the aforesaid biomass. Centrifugation separates the heavy-phase biomass from liquid-phase solution according to their density differences and is preferred to be adopted since less bacteria lost. In all the embodiments of the present invention, the fermented broth is centrifuged at 2000 g-12000 g for 5-60 minutes. The speed and time for the aforesaid centrifugation in use is 12000 g for 20 minutes but some adjustments in speed and time for the aforesaid centrifugation are also acceptable. Longer time or higher speed for centrifuging can increase the harvesting efficiency but with no significant effects on the result of biomass harvesting. The biomass precipitated from the fermented broth is then re-suspended in water or aqueous buffer like dipotassium phosphate solution 110. The concentration of the aforesaid dipotassium phosphate buffer solution in use is above 0 and below or equal to 2M and the volume for washing is 0.5-5 times larger than the volume of the broth collected. A larger volume, ex. five times volume of the collected broth, of the above-said washing buffer is better for the later steps, but buffer of too large volume will increase the time and material cost during conditioning step. The biomass added with the aforesaid water or dipotassium phosphate buffer solution is then stirred for a preferred period of 20-60 minutes and then centrifuged at 2000-12000 g for 5-60 minutes again to precipitate the biomass. The aforesaid period for stirring can be adjusted according to the volume of collected biomass, but stirring for too short time or too much time should be avoided. Stirring for shorter time results in insufficient re-suspension especially when the volume of the biomass is large. Stirring for too much time will cause undesired cell death or degradation. The adjustment in the aforesaid speed and time for the aforesaid centrifugation is also acceptable; longer time or higher speed for centrifuging can increase the harvesting efficiency but still has no significant effects on the result of biomass harvesting.

To obtain a clean biomass which is suitable for the later steps, alcohol of 0.5 to 5 volumes of the ferment broth volume is added slowly to the above-said isolated biomass 120. The above-said alcohol includes methanol, ethanol, isopropanol and their mixtures. A larger volume, ex. five volume of the collected broth volume, of the above-said washing alcohol is better for the later steps, but the above-said washing alcohol of too large volume will increase more time and material cost during conditioning step. After being stirring for 10-120 minutes, the biomass/alcohol mixture is then filtrated and a clean biomass well-conditioned for extraction is obtained. The above step of alcohol treatment can be repeated again to obtain a cleaner biomass more appropriate for extraction and keep the water-content in the wet biomass is below 5%. The aforesaid time for stirring, similarly here, can be adjusted, but stirring for too short time or too much time should be avoided. Stirring for a short time results in insufficient re-suspension, and stirring for too much time will cause lycopene degradation.

Various organic solvents can be used for the extraction of the lycopene from a conditioned E. coli biomass. This invention will refer to the use of solvents that are reasonably high solubility for the lycopene, such as methylene chloride, hexane and acetone. A stream of nitrogen or an antioxidant like BHT is applied to prohibit the occurrence of oxidation of lycopene 130 during extraction. The extraction time will be the minimum which is necessary to achieve solution and is between 1 minute and 60 minutes. To determine the time for extraction can rely on the observation by eyes over the fading change of the orange-red color in the biomass. The amount of the solvent used is determined according to the richness of lycopene in the biomass and varies from 0.5 ml-20 ml of the solvent for per gram wet biomass. The step of extraction can be repeated for 1 to 5 times or washed with the solvent until the color of orange-red biomass disappeared. Commercially available common paper filters are used to separate the liquid phase from the solid phase extract 140. The separated liquid-phase extract is filtrated then by passing through a 0.45 m filter. Other filters of different materials or with different pore sizes are also acceptable as long as these filters are resistant to the solvent in use and efficient to remove the tiny particles derived from the extracted cells 150.

Once the liquid-phase extract by filtration 150 which is rich in lycopene is obtained, it has to be concentrated by using the vacuum 160. The temperature for the step of concentration must be below 40 and preferably below 30 and more preferably below 25. The time for concentration must be less than 8 hours, preferably less than 6 hours, and more preferably less than 4 hour. During this concentrating step, it is not necessary to add the additional precipitating agent. The lycopene will be crystallized gradually during concentration.

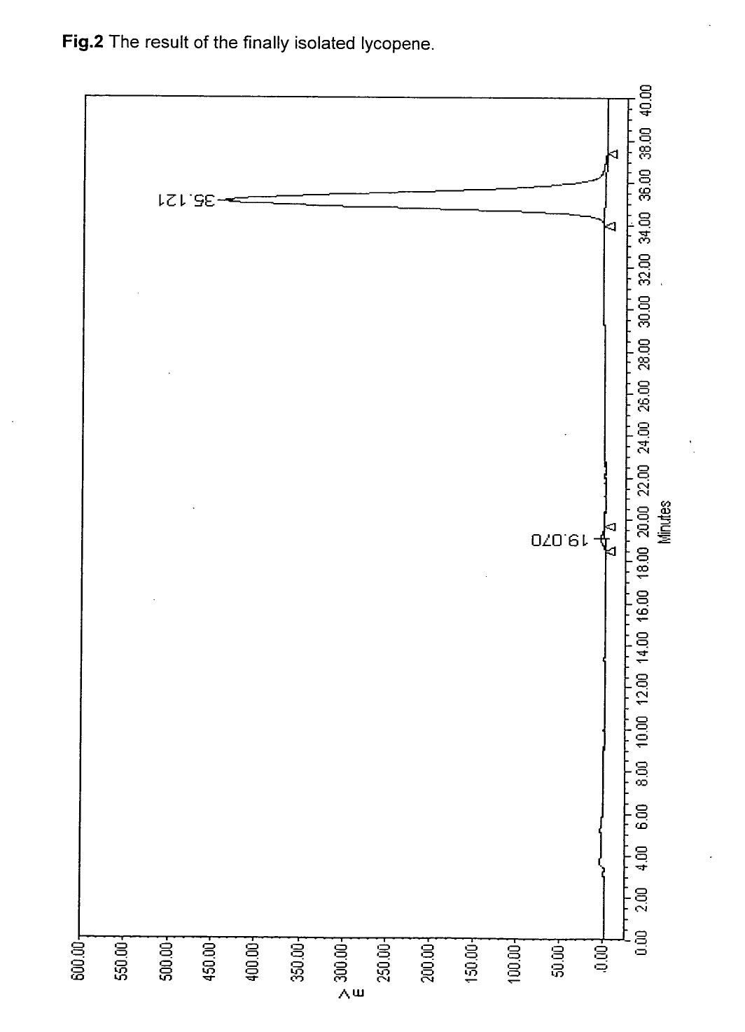

The crystals can be separated from the mother liquor by filtration or centrifugation 170. The separated crystals are then re-suspended in 20-55 acetone for 1-60 minutes 180. The volume of the acetone can be determined according to the ratio of 5 ml to 150 ml acetone for per gram of the above-said wet crystal. The acetone-treated crystal is then collected by using commercially available common paper filters and dried under vacuum 190 until the content of residual solvents meets the specifications and get near all-trans lycopene (FIG. 2). Meeting the requirement of specifications means that these crystals can be used in the food, pharmaceutical or cosmetics industry.

The process described here enables the embodiments with the purity above 92%, preferably above 95% and more preferably of about 99% and with the recovery above 75%, preferably above 80% and more preferably of about 87%. The purity of the crystals obtained by the method described above is determined by the spectrophotometry and by the measurement of the absorption at 472 nm UV light in the purified crystal sample dissolved in n-hexane (E1% 1cm=3450).

The method of this invention is especially suitable for the recovery of crystalline lycopene from a microbial source, preferably bacteria, more preferably from E. coli that contain lycopene synthesis genes.

### EXAMPLE 1

4.18 liters of fermentation broth, after the high-cell density fermentation of the genetically engineered E. coli containing lycopene synthesis genes, are harvested. The strength of the broth is 1.362 g of lycopene per liter. In order to harvest the biomass from the aforesaid broth, the broth is centrifuged at the speed of 12000 g for 20 minutes 100. The harvested biomass is mixed with 4.68 liters of 0.1 M dipotassium phosphate buffer and stirred for 30 minutes 110. Another centrifugation at the speed of 12000 g for 30 minutes is carried out again 110 and the precipitated biomass is then re-suspended in 3.14 liters of isopropanol 120. After being stirred slowly for 60 minutes, the biomass is recovered by centrifugation. A second re-suspension of centrifuged biomass in 2.09 liters of isopropanol is carried out and both the stirring step and centrifuging step are as the same as the steps in the previous isopropanol treatment of this invention 120. A wet biomass weighted 659.1 g is then acquired.

The collected wet biomass is 659.1 g and then extracted by mixing with 1.26 liters of methylene chloride and is stirred under the stream of nitrogen and with the existence of antioxidant (0.32 g BHT) for 30 minutes 130. The liquid-phase extract is separated by filtering the aforesaid methylene chloride/biomass mixture with commercially available common paper filters 140. And the biomass of solid phase retained on the paper filter is washed with about 8 L of methylene chloride 140. These two filtrates of liquid phase extract are combined and the combined extract is filtrated with 0.45 μm filters 150. The aforesaid filtrate is then concentrated under vacuum at the temperature less than 30 160. The extracted lycopene is going to be crystallized during the time the concentration proceeds. The dark red crystals are filtered with commercially available common paper filters 170 and the crystals are re-suspended in 150 ml of 50 acetone for 30 minutes and collected by filtering with commercially available common paper filters 180. The collected dark red crystals are dried under vacuum 190. The dried lycopene crystals have the weight of 4.67 g with a purity of 98.5%. The recovery in this embodied example is 82%.

### EXAMPLE 2

10 liters of fermentation broth after genetic engineered E. coli fermentation production is collected 100. The strength of the broth is 1.284 g of lycopene per liter. In order to harvest the biomass from the aforesaid broth, the broth is centrifuged at the speed of 12000 g for 20 minutes. The harvested biomass is mixed with 10 liters of water and stirring for 30 minutes 110. Another centrifugation at the speed of 12000 g for 30 minutes is carried out again and the precipitated biomass is re-suspended in 10 liters of isoprapanol 120. After stirring for 60 minutes, the biomass is recovered by centrifugation. A second re-suspension of centrifuged biomass in 10 liters of isopropanol is carried out 120 and both the stirring step and centrifuging step are as same as the steps in the previous isopropanol treatment.

The 2821 g collected wet biomass is extracted by mixing with 5.48 liters of methylene chloride and stirring under the stream of nitrogen for 30 minutes & 1.37 g BHT 130. The liquid-phase extract is separated from the biomass of solid phase by filtering with commercially available common paper filters 140. The biomass of solid phase retained on the filter paper is washed with methylene chloride. These two methylene chloride filtrates are combined and filtered with 0.45 μm membrane 150. The filtrated methylene chloride extract is then concentrated under vacuum at the temperature less than 30 160. The extracted lycopene is going to be crystallized during the time the concentration proceeds. The dark red crystals are separated by using commercially available common paper filters 170 and the crystals are then suspended with 150 ml of 50 acetone for stirring 30 minutes 180. After separation by using commercially available common paper filters, the crystals are dried and have the weight of 10.81 g with a purity of 100.4 %. The recovery yield in this embodied example is 84.2%.

The purity and the HPLC analysis of the purified lycopene with acetone treatment are shown in FIG. 2. A Waters Prep 4000 HPLC with a Waters 2487 UV detector (set at 470 nm) was used for lycopene determination. Isocratic separation of unknown samples (20 1 injection volume) was achieved on an YMC C30 (3 m) (4.6×250 mm) column. The mobile phase methanol/methyl-t-butyl ether (50:50) was perfused at a flow rate of 1.0 ml/min.

What is claimed is:

1. A process for obtaining the biosynthesized lycopene from transformed or naturally derived bacterial cells, which comprises the steps of:
(a) isolating a biomass from a fermented broth;
(b) treating said isolated biomass by adding alcohol and isolating said alcohol-treated biomass;
(c) extracting the biosynthesized lycopene from said alcohol-treated biomass by adding methylene chloride;
(d) removing the solid phase from biomass/methylene chloride extracting mixture to obtaining a liquid phase extract;
(e) filtering said liquid-phase extract;
(f) crystallizing the lycopene by concentrating said liquid-phase extract under vacuum;
(g) separating the lycopene crystals;
(h) re-suspending said separated lycopene crystals with acetone and separating lycopene crystals from said acetone; and
(i) drying said acetone-treated lycopene crystals.

2. The process of claim 1, wherein said transformed bacterial cells are transformed Escherichia coli cells.

3. The process of claim 1, further comprising a step of washing biomass with water or buffer like potassium phosphate dibasic buffer before said step of (b).

4. The process of claim 1, wherein said alcohol comprises methanol.

5. The process of claim 1, wherein said alcohol comprises ethanol.

6. The process of claim 1, wherein said alcohol comprises isopropanol.

7. The process of claim 1, an antioxidant or a stream of nitrogen is applied in said step (c).

8. The process of claim 1, wherein said liquid-phase extract in said step (e) is filtered by using a 0.45 μm filter membrane.

9. The process of claim 2, further comprising a step of washing biomass with water or buffer like potassium phosphate dibasic buffer before said step of (b).

10. The process of claim 2, wherein said alcohol comprises methanol.

11. The process of claim 2, wherein said alcohol comprises ethanol.

12. The process of claim 2, wherein said alcohol comprises isopropanol.

13. The process of claim 2, an antioxidant or a stream of nitrogen is applied in said step (c).

14. The process of claim 2, wherein said solid phase in said step (e) is removed by using a 0.45 μm filter membrane.

15. Biosynthesized lycopene produced according to the process of claim 1.

16. Biosynthesized lycopene produced according to the process of claim 2.

17. Biosynthesized lycopene produced according to the process of claim 3.

18. Biosynthesized lycopene produced according to the process of claim 9.
